(19) United States
(12) Patent Application Publication
(10) Pub. No.: US 2004/0038958 A1
(43) Pub. Date: Feb. 26, 2004

(54) Topical treatment of skin diseases

(76) Inventors: Chris Rundfeldt, Coswig (DE); Manfred Kietzmann, Grossburgwedel (DE); Joachim Hoppmann, Radebeul (DE); Wolfgang Baumer, Hannover (DE); Hildegard Kuss, Dresden (DE); Norbert Hofgen, Ottendorf-Okrilla (DE)

Correspondence Address: FULBRIGHT & JAWORSKI, LLP, 666 FIFTH AVE, NEW YORK, NY, 10103-3198, US

(21) Appl. No.: 10/611,649

(22) Filed: Jul. 1, 2003

Related U.S. Application Data
(60) Provisional application No. 60/395,221, filed on Jul. 11, 2002.

(51) Int. Cl. A61K 31/541; A61K 31/5377; A61K 31/496; A61K 31/454; A61K 31/427; A61K 31/422; A61K 31/4178

(52) U.S. Cl. 514/210.21; 514/228.2; 514/232.8; 514/254.09; 514/323; 514/365; 514/374; 514/397; 514/411; 514/414; 514/419

(57) ABSTRACT

The present invention relates to a method for the treatment of an inflammatory and/or allergic skin disease comprising topically administering a substituted hydroxy indol.

### DESCRIPTION

The present invention relates to a method for the treatment of an inflammatory and/or allergic skin disease comprising topically administering a substituted hydroxy indol.

Phosphodiesterase (PDE) isoenzymes are involved in the regulation of cellular signal transduction cascades by the modulation of cyclic nucleotide levels. To date 11 PDE isoenzyme gene families have been identified (Giembycz, 2000). These isoenzymes differ in their cellular distribution and biochemical function. In leukocytes of patients with atopic dermatitis, in particular in children, a high PDE4 activity was found (Butler et al., 1983; Cooper et al., 1985). PDE4 is a major isoenzyme in inflammatory cells, such as monocytes and monocyte derived macrophages (Gantner et al., 1997), eosinophils (Dent et al., 1994) and B lymphocytes (Cooper et al., 1985). PDE4 inhibitors exhibit very strong anti-inflammatory effects by an increase of the intracellular cAMP level. Via an inhibition of cAMP degradation PDE4 inhibitors modulate intracellular functions (e.g. attenuation of superoxide generation) and gene transcription (e.g. inhibition of synthesis and/or release of inflammatory cytokines) (Giembycz, 2000, Kuss et al., 2002). As PDE4 is also expressed in keratinocytes, these cells may be an additional potential pharmacologic target for the control of inflammatory disorders in the skin using PDE4 inhibitors (Chujor et al., 1998).

Hydroxy indols, their use as inhibitors of PDE4 and methods for their preparation are disclosed in WO99/55696. These compounds can be employed in disorders which are associated with the activity of eosinophils, particularly inflammatory airway disorders, such as bronchial asthma, allergic rhinitis, allergic conjunctivitis, atopic dermatitis, eczema, allergic angiitis, inflammations and proliferative skin disorders, such as psoriasis or keratosis. Possible administration forms for these compounds are oral, parenteral, intravenous, transdermal, topical, inhalational and intranasal preparations. An especially preferred compound is AWD12-281.

The PDE4 inhibitor AWD 12-281 (N-(3,5-Dichloro-4-pyridinyl)-2-[1-(4-fluoro-benzyl)-5-hydroxy-1 H-indol-3-yl]-2-oxoacetamide) was successfully tested in a model of allergic bronchoconstriction. It significantly reduced the bronchospasmogenic effect of an allergen in passively sensitized human airways (Schmidt et al., 2000). AWD 12-281 inhibited the release of inflammatory mediators in antigen stimulated human cells from nasal polyps such as GM-CSF (granulocyte-macrophage colony-stimulating factor), TNF-α (tumor necrosis factor α) and histamine (Kuss et al., 2002). Additionally, AWD 12-281 inhibited the degranulation of human eosinophils in vitro (Ezeamuzie, 2001). In vivo, AWD 12-281 significantly reduced the accumulation of eosinophils in bronchoalveolar lavage in the late phase airway reaction to antigen in sensitized Brown Norway rats. It also showed inhibitory effects in LPS induced lung neutrophilia in domestic pigs (Kuss et al., 2002).

A further PDE4 inhibitor is cilomilast which is currently evaluated for the treatment of asthma (Griswold et al., 1998, Giembycz, 2000) and chronic obstructive pulmonary disease. Particularly the Phase II/III clinical trials concerning chronic obstructive pulmonary disease have demonstrated a clinically significant increase in lung function (Giembycz, 2001, Dyke & Montana, 2002).

To stimulate an immunological inflammation, the already established (Ehinger et al., 2000) mouse ear swelling test (MEST) with mice sensitized according to Gad et al. (1986) to toluene-2,4-diisocyanate (TDI) was used. It was demonstrated by Dearman et al. (1996) that TDI sensitization of the skin leads to a Th2-type cytokine production in BALB/c mice. Activated lymph node cells from TDI exposed animals produce substantial amounts of interleukin 4 and 10 but only low levels of interferon γ. Depending on the sensitization regime, the contact allergen TDI induces an IgE-independent (short exposure) or IgE-dependent (long exposure) allergic dermatitis (Scheerens et al., 1999). As high IgE values are only obtained by a long exposure sensitization In one study a sensitization was carried out over 120 days.

Studies were performed to gain information about the effectiveness of different PDE4 inhibitors for their therapeutical effect on allergic and/or inflammatory reactions. To improve the characterization of TDI induced ear swelling, the cytokines interleukin (IL) 4, IL-6 and MIP-2 were measured in treated mouse ears.

It was found that topical administration of AWD12-281 after the TDI challenge as therapeutic intervention caused a significant inhibition of ear swelling. In contrast thereto, the PDE4 inhibitor cilomilast (SB207499), which was taken as a reference compound failed to do so. This result indicates that topically administered hydroxyindols, such as AWD12-281 are potent in the prevention and treatment of allergic or inflammatory skin diseases.

Thus, the present invention relates to a method for the treatment of a skin disease comprising topically administering a subject in need thereof therapeutically effective amounts of a compound of formula (1) or a pharmacologically acceptable salt thereof:

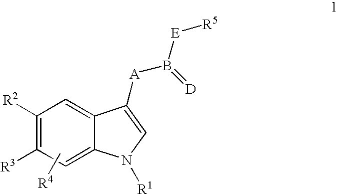

in which

R¹ is

(i) —C₁₋₁₂-alkyl, straight-chain or branched-chain or —C₂-C₁₂ alkenyl, mono- or polyunsaturated,

optionally mono- or polysubstituted by —OH, —SH, —NH₂, —NHC₁₋₆-alkyl, —N(C₁₋₆alkyl)₂, —NHC₆₋₁₄aryl, —N(C₆₋₁₄aryl)₂, —N(C₁₋₆alkyl)(C₆₋₁₄aryl), —NHCOR⁶—NO₂, —CN, —F, —Cl, —Br, —I, —O—C₁₋₆-alkyl, —O—C₆₋₁₄-aryl, —O(CO)R⁶, —S—C₁₋₆-alkyl, —S—C₆₋₁₄aryl, —SOR⁶, —SO₃H, —SO₂R⁶—OSO₂C₁₋₆alkyl, —OSO₂C₆₋₁₄aryl, —(CS)R⁶, —COOH, —(CO)R⁶, mono-, bi- or tricyclic saturated or mono- or polyunsaturated carbocycles having 3-14 ring members, mono-, bi- or tricyclic saturated or mono- or polyunsaturated heterocycles having 5-15 ring members and 1-6 heteroatoms, which are preferably N, O and S, wherein the C₆₋₁₄ aryl groups and the carbocyclic and heterocyclic substituents can optionally be mono- or polysubstituted by R⁴,

(ii) a mono-, bi- or tricyclic saturated or mono- or polyunsaturated carbocycle having 3-14 ring members or a mono-, bi- or tricyclic saturated or mono- or polyunsaturated heterocycle having 5-15 ring members and 1-6 heteroatoms, which are preferably N, O and S, or a carbo- or heterocyclic saturated or mono- or polyunsaturated spirocycle having 3-10 ring members, where heterocyclic systems contain 1-6 heteroatoms, which are preferably N, O and S,

optionally mono- or polysubstituted by —OH, —SH, —NH₂, —NHC₁₋₆-alkyl, —N(C₁₋₆-alkyl)₂, —NHC₆₋₁₄aryl, —N(C₆₋₁₄aryl)₂, —N(C₁₋₆alkyl)(C₆₋₁₄aryl), —NHCOR⁶, —NO₂, —CN, —F, —Cl, —Br, —I, —O—C—₁₋₆-alkyl, —O—C₆₋₁₄-aryl, —O(CO)R⁶, —S—C₁₋₆-alkyl, —S—C₆₋₁₄aryl, —SOR⁶, —SO₃H, —SO₂R⁶, —OSO₂C₁₋₆alkyl, —OSO₂C₆₋₁₄aryl, —(CS)R⁶, —COOH, —(CO)R⁶, mono-, bi- or tricyclic saturated or mono- or polyunsaturated carbocycles having 3-14 ring members, mono-, bi- or tricyclic saturated or mono- or polyunsaturated heterocycles having 5-15 ring members and 1-6 heteroatoms, which are preferably N, O and S, wherein the C₆₋₁₄aryl groups and the carbocyclic and heterocyclic substituents can optionally be mono- or polysubstituted by R⁴,

R⁵ is

a mono-, bi- or tricyclic saturated or mono- or polyunsaturated carbocycle having 3-14 ring members or a mono-, bi- or tricyclic saturated or mono- or a polyunsaturated heterocycle having 5-15 ring members and 1-6 hetero-atoms, which are preferably N, O and S, or a carbo- or heterocyclic saturated or a mono- or polyunsaturated spirocycle having 3-10 ring members, where heterocyclic systems contain 1-6 heteroatoms, which are preferably N, O and S,

optionally mono- or polysubstituted by —OH, —SH, —NH₂, —NHC₁₋₆-alkyl, —N(C₁₋₆-alkyl)₂, —NHC₆₋₁₄aryl, —N(C₆₋₁₄aryl)₂, —N(C₁₋₆alkyl)(C₆₋₁₄aryl), —NHCOR⁶, —NO₂, —CN, —F, —Cl, —Br, —I, —O—C₁₋₆-alkyl, —O—C₆₋₁₄-aryl, —O(CO)R⁶, —S—C₁₋₆-alkyl, —S—C₆₋₁₄aryl, —SOR⁶, —SO₃H, —SO₂R⁶, —OSO₂C₁₋₆alkyl, —OSO₂C₆₋₁₄aryl, —(CS)R⁶, —COOH, —(CO)R⁶, mono-, bi- or tricyclic saturated or mono- or polyunsaturated carbocycles having 3-14 ring members, mono-, bi- or tricyclic saturated or mono- or polyunsaturated heterocycles having 5-15 ring members and 1-6 heteroatoms, which are preferably N, O and S, wherein the C₆₋₁₄aryl groups and the carbocyclic and heterocyclic substituents can optionally be mono- or polysubstituted by R⁴,

with the proviso that R⁵ contains at least one substituent selected from —F, —Cl, —Br, —I:

R², R³ are hydrogen or —OH, where at least one of the two substituents must be —OH;

R⁴ is

—H, —OH, —SH, —NH₂, —NHC₁₋₆-alkyl, —N(C₁₋₅-alkyl)₂, —NHC₆₋₁₄aryl, —N(C₆₋₁₄aryl)₂, —N(C₁₋₆alkyl)(C₆₋₁₄aryl), —NHCOR⁶, —NO₂, —CN, —COOH, —(CO)R⁶, —(CS)R⁶, —F, —Cl, —Br, —I, —O—C₁₋₆-alkyl, —O—C₆₋₁₄-aryl, —O(CO)R⁶, —S—C₁₋₆-alkyl, —S—C₆₋₁₄aryl, —SOR⁶, —SO₂R⁶, —C₁—C₆-alkyl, wherein each aryl or alkyl may be mono- or polysubstituted by —OH, —F, —Cl, —Br, —I;

R⁶ is

—H, —NH₂, —NHC₁₋₆-alkyl, —N(C₁₋₆-alkyl)₂, —NHC₆₋₁₄aryl, —N(C₆₋₁₄aryl)₂, —N(C₁₋₆alkyl) (C₆₋₁₄aryl), —O—C₁₋₆-alkyl, —O—C₆₋₁₄-aryl, —S—C₁₋₆-alkyl, —S—C₆₋₁₄aryl, —C₁₋₁₂-alkyl, straight-chain or branched-chain,

—C₂₋₁₂-alkenyl, mono- or polyunsaturated, straight-chain or branched-chain, mono-, bi- or tricyclic saturated or mono- or polyunsaturated carbocycles having 3-14 ring members,

mono-, bi- or tricyclic saturated or mono- or polyunsaturated heterocycles having 5-15 ring members and 1-6 heteroatoms, which are preferably N, O and S;

A is either a bond, or

—(CH₂)ₘ—, —(CH₂)ₘ—(CH═CH)ₙ—(CH₂)ₚ—, —(CHOZ)ₘ—, —(C═O)—, —(C═S)—, —(C═N—Z)—, —O—, —S—, —NZ—,

wherein m, p=0-3 and n=0-2 and

Z is

—H, or

—C₁₋₁₂-alkyl, straight-chain or branched-chain,

—C₂₋₁₂-alkenyl, mono- or polyunsaturated, straight-chain or branched-chain, mono-, bi- or tricyclic saturated or mono- or polyunsaturated carbocycles having 3-14 ring members,

mono-, bi- or tricyclic saturated or mono- or polyunsaturated heterocycles having 5-15 ring members and 1-6 heteroatoms, which are preferably N, O and S;

B is either carbon or sulfur, or —(S═O)—;

D is oxygen sulfur, CH₂ or N—Z,

where, if B is carbon, D is S or CH₂;

E is a bond, or

—(CH₂)ₘ—, —O—, —S—, —(N—Z)—, wherein m and Z have the meaning already described above.

The invention furthermore relates to the pharmaceutically acceptable salts of the compounds according to formula (I).

In the compounds of formula (I) R⁵ is preferably selected from monocyclic saturated or mono- or polyunsaturated carbocycles and heterocycles having at least one halogen-substituent, more preferably from monocyclic aromatic carbocycles and heterocycles having at least one, e.g. 2 or 3 halogen substituents. Especially preferred examples of R⁵ are pyridine or phenyl rings having at least one halogen substituent, such as 3,5-dichloro-4-pyridyl, 2,6-dichlorophenyl, 2,6-dichloro-4-trifluoromethylphenyl, 2,6-dichloro-4-trifluoromethoxy phenyl etc.

R¹ is preferably selected from C₁-C₁₂ alkyl, e.g. C₁-C₄ alkyl, which is optionally substituted by a carbocyclic ring, e.g. by a phenyl ring. Especially preferred examples of R¹ are ethyl, propyl (n-propyl or isopropyl), benzyl and halogen-substituted benzyl, such as 4-fluorobenzyl, 2,6-difluorobenzyl etc. Furthermore, R¹ may be selected from monocyclic saturated or mono- or polyunsaturated carbocycles or heterocycles; which are optionally substituted.

R² is preferably OH and R³ is preferably H. A is preferably selected from —(C═O)— and —(CHOH)—. B is preferably C, C is preferably O and E is preferably —(N—H)—. An especially preferred example of a compound (I) is AWD 12-281 (N-3,5-dichloro-4-pyridinyl)-2 [1-(4-fluorobenzyl)-5-hydroxy-1 H-indol-3-yl]-2-oxoacetamide) or a pharmaceutically acceptable salt thereof.

The compounds of the invention are particularly suitable for the treatment of an inflammatory and/or allergic skin disease, more particularly a skin disease associated with a pathologically increased PDE4-activity, for example allergic diseases, such as allergic dermatitis.

In general, the compounds of formula (I) or pharmaceutically acceptable salts thereof can be used in the manufacture of a medicament for the prophylactic or therapeutic treatment of any disease state in humans, or other mammals, which is caused by inflammatory reactions of the skin, as well as disturbed proliferation and differentiation of dermal cells. The compounds of formula (I) may be used in the treatment of inflammatory reactions of the skin like scleritis, sclerodermia circumscripta, erysipelas, pemphigus vulgaris, pemphigus foliaceus and bullus pemphigoid. The compounds also have a beneficial effect on inflammatory skin diseases (e.g. lichen ruber planus) caused by activated T-cells or other cells of the immune system, like granulocytes, mast-cells, macrophages, or released mediators of these immune cells, like cytokines. Furthermore these compounds are active in the treatment of skin manifestations of auto-immunological diseases, like dermatomyositis, lupus erythrematosus etc.

As these compounds are able to elevate cyclic AMP levels, they are therefore of use in the treatment of benign and malignant proliferative skin diseases in human or non-human mammals. When used herein the expression ‘proliferative skin diseases’ means benign and malignant proliferative skin diseases which are characterized by accelerated cell division in the epidermis, dermis or appendages thereto, associated with incomplete tissue differentiation. Such diseases include: psoriasis, atopic dermatitis, non-specific dermatitis, primary irritant contact dermatitis, allergic contact dermatitis, or allergic disorders such as atopy, urticaria, eczema, kerato-conjunctivitis, basal and squamous cell carcinomas of the skin, lamellar ichthyosis, epidermolytic hyperkeratosis, epidermolysis bullosa simplex, premalignant sun induced keratosis, non-malignant keratosis, acne, and seborrheic dermatitis, Lyell syndrome, granulomatous skin lesions in humans and atopic dermatitis, pruritis and mange in domesticated animals.

As the compounds are able to reduce inflammatory reactions, they are useful in the treatment of skin diseases, which are induced by infection with bacterial, virus, fungus or parasites Examples for these diseases are erythema migrans, tuberculosus cutis, lyme-disease, dermal leishmaniasis, toxic epidermal necrolysis, pyodermas, tinea and haemorrhoids,

The compounds of formula (I) may also be used in the treatment of inflammatory reactions of the skin like scleritis, sclerodermia circumscripta, erysipelas, pemphigus vulgaris, pemphigus foliaceus and bullus pemphigoid.

As proliferation of dermal cells is affected through intracellular cAMP levels, a compound of formula (I) support wound healing.

The compounds (I) are administered as topical, e.g. transdermal, formulations, preferably in form of aqueous or oily suspensions containing the active ingredient and suitable pharmaceutically acceptable carriers, diluents and adjuvants.

In general, compounds of formula (I), or if appropriate pharmaceutically acceptable salts thereof, may be administered as a topical formulation in combination with conventional topical excipients. Topical formulations may be presented as, for instance, ointments, pastes, linements, drops, creams or lotions, impregnated dressings, gels, gel sticks, spray and aerosols, and may contain appropriate conventional additives such as preservatives, solvents to assist drug penetration and emollients in ointments and creams. The formulations may contain compatible conventional carriers, such as cream or ointment bases and ethanol or oleyl alcohol for lotions. Suitable cream, lotion, gel, stick, ointment, spray or aerosol formulations that may be used for compounds of formula I or if appropriate a pharmaceutically acceptable salt thereof. If a treatment of rectal and anal skin resp. mucosa is indicated, compounds of formula (I) may be administered as suppositories.

The dosage of the active compounds can vary depending on the age and weight of the patient, nature and severity of the disease to be treated and similar factors. The daily dosage can be administered as an individual dose or subdivided into two or more daily dosages and may be in the range of 0.01-5000 mg.

In an especially preferred embodiment of the present invention, the compound is administered to a skin area, which is already afflicted by disease, For example, the compound is administered after an allergic challenge, i.e. after the patient to be treated has been exposed to an allergen and preferably after the first allergic symptoms are observed. Particularly, the first administration of the compound may be up to 48 h, preferably up to 24 h after an allergic challenge. Then, the administration will continue until the desired effect has been obtained.

The compound (I) may be administered as sole active ingredient or in combination with at least one further pharmaceutical agent. For example, the compounds of formula (I) can be combined with drugs stimulating cAMP production, for example sympathomimetic amines such as isoprenaline, isoetharine, salbutamol, phenylephrine and ephedrine; xanthine derivatives such as theophylline and aminophylline and corticosteroids such as prednisolone and adrenal stimulants such as ACTH may be included. A combination of pharmaceutical agents which possess an influence on the immune system like steroids, immune-suppressants (e.g. tacrolimus, sirolimus, cyclosporine, pimecrolimus), cyclooxygenase inhibitors (e.g. indomethacin, diclofenac, ibuprofen), or antihistamines (diphenhydramine, hydroxyzine, loratadine, cetirizine). The compound of formula (I) may, be administered concurrently with other agents useful for the treatment or management of skin diseases like retinoids, dithranol, vitamin D derivates, alefacept, daclizumab, etanercept and the like. Treatment of skin diseases which are caused by infections with bacterial, virus, fungus or parasites, may be supported with antibiotics like sulfonamides, erythromycin and tetracyclines. As proteins like chemokines (IP-10) or cytokines (e.g. IL-1β, IL-2) influence the differentiation and proliferation of dermal cells, a supplementation with these mediators or with antibodies may posses a benefit for treating skin diseases.

Further, the present invention shall be explained in more detail by the following Figures and Examples.

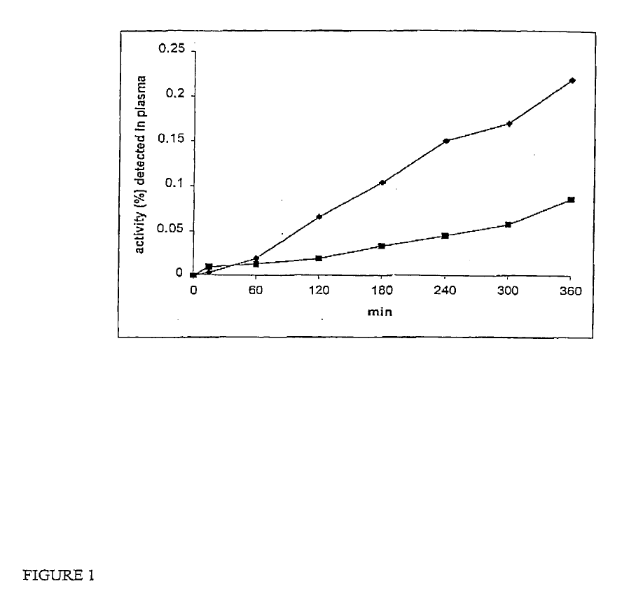

FIG. 1: Cutaneous permeation of ¹⁴C AWD 12-281 measured using a “Franz” diffusion cell and murine back skin. Activity of ¹⁴C AWD 12-281 was measured in plasma during 360 min incubation. Results of two independent experiments. 15 μl (110322970 dpm) of ¹⁴C AWD 12-281 (dissolved in acetone/DMSO 1:1) was applied to the shaved skin.

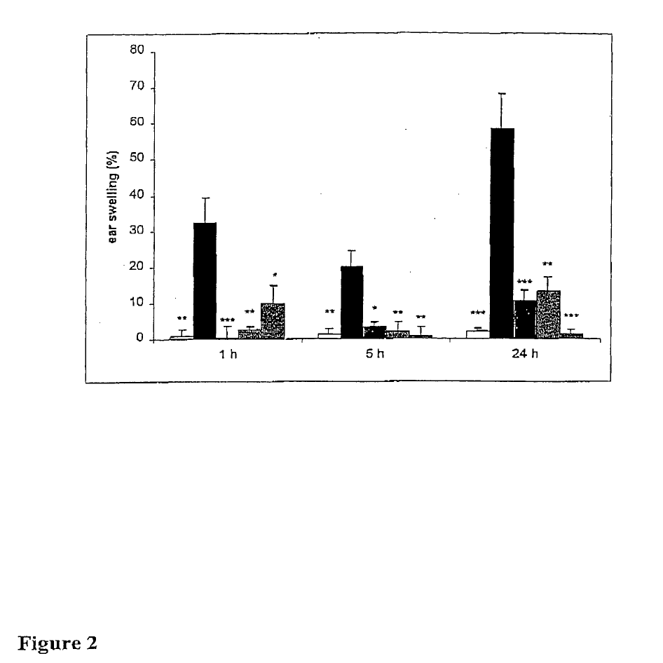

FIG. 2: Effect of a single topical application AWD 12-281, cilomilast and diflorasone onto the ears of mice sensitized to TDI two hours before TDI-challenge. Bars represent ear swelling 1, 5 and 24 h after related to values obtained before TDI challenge. There is a significant increase of the ear swelling in TDI treated mice (black bars) compared to untreated controls (white bars). AWD 12-281(1%, vertically hatched bars) as well as cilomilast (3%, cross hatched bars) and diflorasone (0.05%, grey bars) inhibited the swelling significantly at all measured times. *P<0.05, **P<0.01, ***P<0.001 in comparison to TDI treated animals (n=6 (TDI=12) each group).

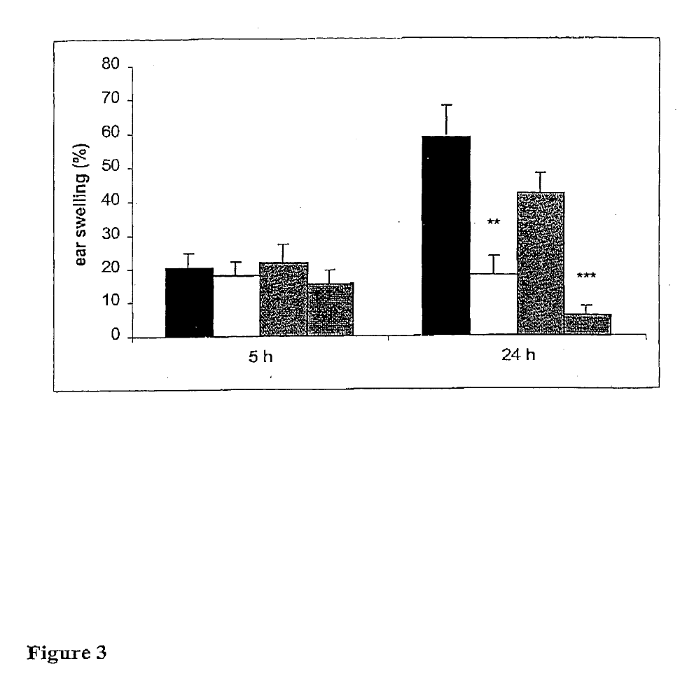

FIG. 3: Effect of a single topical application of AWD 12-281, cilomilast and diflorasone onto the ears of mice sensitized to TDI one hour after TDI-challenge (therapeutic intervention). Bars represent earswelling 5 and 24 h after TDI challenge, There was no significant difference between the treated groups 5 h after the challenge. Compared to TDI challenged control mice, AWD 12-281 (1%, white bars) as well as diflorasone so (0.05%, grey bars) inhibited the swelling significantly 24 h after the challenge. Cilomilast (3%, cross hatched bars) caused no significant inhibition. **P<0.01, (n=6 (TDI=12) each group).

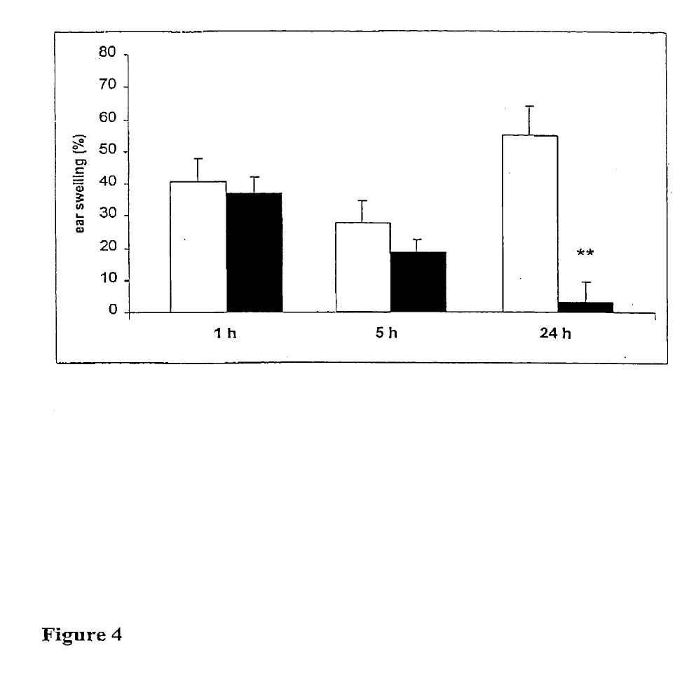

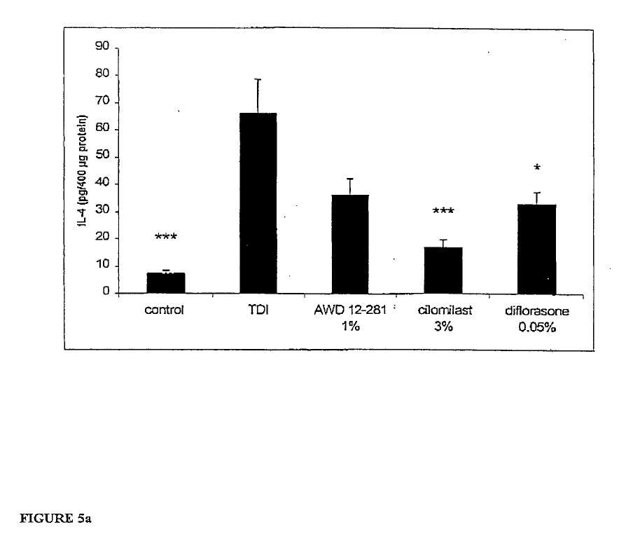

FIG. 4: Effect of AWD 12-281 on TDI induced ear swelling in mice sensitized to TDI using a long term (120 days) repeated exposure sensitization paradigm. Bars represent ear swelling 1, 5 and 24 h after TDI challenge. There was no significant difference between the groups 1 and 5 h after the challenge. Compared to TDI treated control mice (white bars), AWD 12-281 (3%, black bars) administered 1 h after TDI challenge inhibited the swelling significantly 24 h after the challenge. **P<0.01, (n=5 each group).

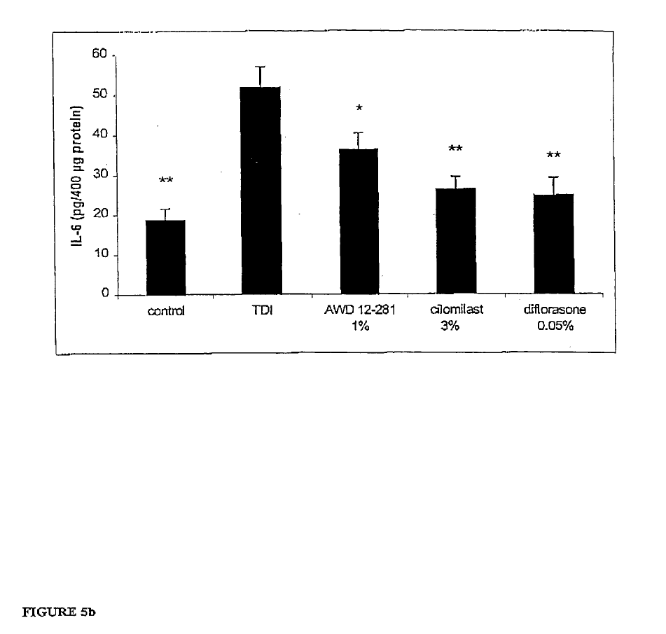

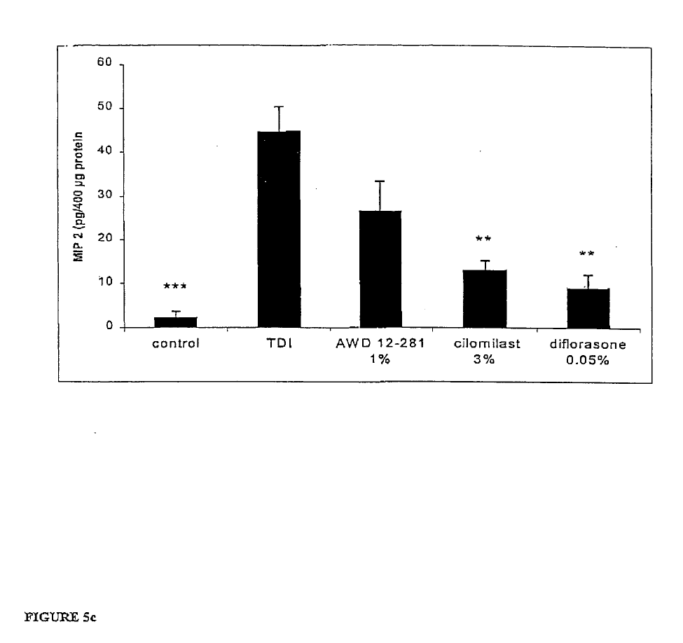

FIG. 5: Concentration of interleukin 4 (A), interleukin 6 (B) and macro-phage inflammatory protein 2 (C) in homogenized mouse ears 24 h after TDI challenge. Samples were taken from the experiment with topical treatment (see FIG. 2). There was a significant increase of interleukin 4, interleukin 6 and macrophage inflammatory protein 2 in TDI challenged mouse ears. AWD 12-281 (1%) reduced the increase slightly (IL-4, MIP-2) or weak significantly (IL-6). Cilomilast (3%) as well as diflorasone (0.05%) reduced the concentration of all mediators significantly. *P<0.05, **P<0.01, ***P<0.001 in comparison with TDI treated animals (n=6(TDI=12) each group).

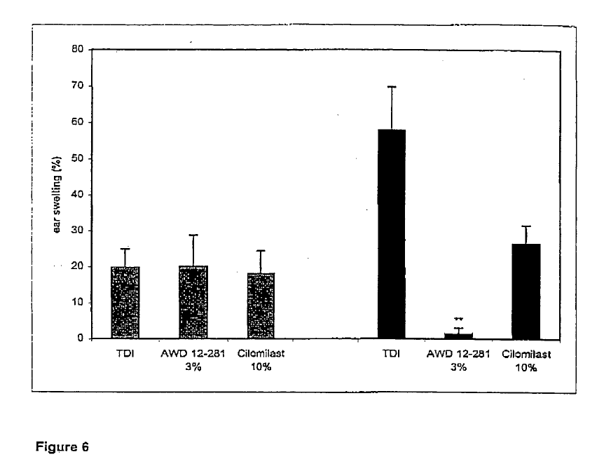

FIG. 6: Effect of PDE 4 inhibitors on TDI induced ear swelling in mice. Mice ears were treated with 0.5% TDI. After 1 h (grey bars) groups of mice were treated with 3% AWD 12-281, 10% cilomilast or left untreated (TDI). After 24 h, ear swelling was determined (black bars). Whereas AWD 12-281 reduced ear swelling significantly cilomilast did not show a significant inhibitory effect (p=0.09).

### EXAMPLE

1. Material and Methods

1.1 Sensitization Procedure

Female BALB/c-mice were obtained from Charles River (Sulzfeld, Germany) at the age of 8 weeks (20 g body weight). All animals were healthy and were housed in groups of six mice per cage at 22° C. with a 12-h light/dark-cycle. Water and a standard diet (Altromin, Lage/Lippe, Germany) were available ad libitum.

After settling in for 1 week, the abdominal skin of the mice was shaved and depilated with Veet®. Subsequently, the horny layers of the abdominal skin were stripped off ten times with adhesive tapes. For active sensitization, 100 μl 5% TDI in acetone were administered to the stripped epidermis on 4 consecutive days.

21 days later, the allergic reaction was boostered by administration of 10 μl 0.5% TDI in acetone on both, the inner and outer surface of the left ears to examine the sensitization status. Before as well as 24 h after challenge, the ear thickness was measured with a cutimeter (model 7309, Mitutoyo, Neuss, Germany) The swelling was calculated by comparison of the values before challenge with 24 h after challenge. Animals that had a mean swelling difference of less than 20% 24 h after challenge compared to the earlier assessed individual basal value (ca. 230 μm) were excluded as being not sensitized. The other mice were equally distributed to the treatment groups (n=6) according to their swelling intensity, so that each group contained animals which had responded to varying extents. They had to rest until the ear thickness had reached almost a normal level after 7 days. To exclude residues of the allergen on the ears, the untreated right ears were used for the main experiment.

1.2 Topical Application

One group of mice (n=6) was not sensitized and challenged. A second group (n=12) was challenged topically by administration of 20 μl (10 μl on both, the inner and outer surface) 0.5% TDI in acetone to the right ears. Two hours before TDI-challenge, the third and fourth group (each n=6) were treated with 20 μl (10 μl on both, the inner and outer surface) AWD 12-281 (1%=200 μg) or 20 μl cilomilast (3%=600 μg; each in acetone/DMSO 9:1) to the right ears. Diflorasone (20 μl, 0.05%=10 μg in acetone) served as a corticoid positive control (n=6). These mice were sacrificed by cervical dislocation 24 hours after TDI challenge and the ears were collected (see below).

To simulate therapeutic conditions, three additional groups (n=6) received AWD 12-281 (1%), cilomilast (3%) or diflorasone (0.05%) topically 1 h after TDI challenge, i.e. directly after measurement of the ear thickness. The ear thickness was determined also 5 and 24 h after TDI-challenge.

Five weeks after the first challenge, the animals treated with AWD 12-281 (1%) and cilomilast (3%) “therapeutically” were challenged again. Before challenge, one group was treated with AWD 12-281 (3%).

Furthermore, an experiment was carried out to obtain information on the therapeutic effects of AWD 12-281 in a model of long term exposure to TDI. Thus 10 mice were treated with 100 μl 0.5% TDI on the abdominal skin in intervals of 10 days for 120 days. Ten days after the last abdominal TDI treatment, mice were challenged on the left ear with 20 μl 0.5% TDI, split in 10 μl onto the outer and inner surface of the ear, respectively. One hour after TDI challenge 5 mice were treated with AWD 12-281 (3%), 5 mice were sham treated. Before as well as 1, 5 and 24 h after challenge, the ear thickness was measured.

1.3 Determination of Cytokines

One part of the collected ears was fixed in 4% formaldehyde for histological section and stained with haematoxylin-eosin with respect to dermal thickness and granulocyte accumulation. These parameters were measured in 10 fields at 40 times magnification. The remaining tissue was shock-frozen and stored in liquid nitrogen immediately after sampling. For the determination of biochemical parameters, the mice ears were homogenized under liquid nitrogen. The homogenates were taken in 200 μl RPMI 1640 medium and the protease inhibitor Pefabloc® (1 mmol) was added and the samples were mixed intensively. After centrifugation (10000 g, 10 min, 4° C.), the supernatant was collected and the protein content was determined. The samples were stored at −80° C. until the cytokines were determined. Interleukin (IL) 4, IL-6 and MIP-2 were measured in the samples by ELISA using commercially available kits according to manufacturers instructions.

1.4 Penetration of ¹⁴C AWD 12-281 Through Murine Back Skin

The ability of ¹⁴C AWD 12-281 to penetrate through murine back skin was tested in a diffusion cell (Franz cell). Dry shaved murine back skin was set onto the diffusion cell so that 1.5 cm (diameter) of the dermal side were in contact with warmed (34° C.) buffer (bovine serum). 15 μl (110322970 dpm) of ¹⁴C AWD 12-281 (dissolved in acetone/DMSO 1:1) were applied to the shaved skin. Samples were taken 15, 60, 120, 180, 240, 300, 360 min and the radioactivity was measured in a β counter (Beckman, Munich, Germany). The experiment was performed twice.

1.5 Reagents

TDI was supplied by Sigma-Aldrich Chemie (Deisenhofen, Germany). AWD 12-281, ¹⁴C AWD 12-281 and cilomilast were obtained from AWD (Dresden, Germany). Acetone, PEG200 and DMSO were purchased from Merck (Darmstadt, Germany); formaldehyde solution from Fluka (Deisenhofen, Germany), Miglyol and hydroxylethylcellulose from Caesar & Loritz (Hilden, Germany) and RPMI 1640 medium from Biochrom (Berlin, Germany). The ELISAs for the determination of the cytokines were purchased from R&D Systems (Wiesbaden, Germany), Pefabloc® was purchased from Boehringer Mannheim (Germany). The depilation cream (Veet®) is a trademark of Reckitt & Colman (Hamburg, Germany). The adhesive tape (Tesafilm®) was obtained from Beiersdorf (Hamburg, Germany). The protein content was measured with a Biorad® assay (Munchen, Germany).

1.6 Statistical Evaluation

Results are presented as mean and standard error (SE). The different treatment groups were checked for significant differences by means of the Mann-Whitney test (U-test). As TDI treated control mice were compared to three to five different groups the number of TDI treated mice was doubled in most experiments (n=12).

2. Results

2.1 Penetration of ¹⁴C AWD 12-281 Through Murine Back Skin

In both experiments an increase of radioactivity was measured in the buffer indicating cutaneous penetration of AWD 12-281. The amount of radioactivity measured 360 min after application resembled 0.22 and 0.08% of the total activity (FIG. 1).

2.2 Topical Administration

Mouse Ear Swelling

The control mice showed a mean increase of about 30%, 20% and 60% in ear thickness 1 h, 5 h and 24 h after the TDI-challenge. When administered 2 h before TDI challenge, topically administered AWD 12-281 (1%) cilomilast (3%) and diflorasone (0.05%) inhibited the TDI-induced swelling significantly at all measured times (FIG. 2)

The groups used to test the therapeutic effect of AWD 12-281 showed a swelling between 25-30% 1 hour after TDI challenge, i.e. directly prior to drug administration. After drug administration, five hours after TDI challenge, no significant further swelling was observed and the different treatment groups did not differ significantly. However 24 h after challenge, AWD 12-281 as well as diflorasone inhibited the TDI induced ear swelling significantly. Cilomilast induced a slight, but not significant reduction of the swelling (FIG. 3).

In the last experiment with long term (120 day) sensitization, application of TDI onto the ears induced an increase of ear thickness of nearly 40% 1 h after challenge. Topically administered AWD 12-281 (3%), applied “therapeutically” 1 h after challenge, induced a slight inhibition 5 h after the challenge. Compared to the untreated control group, 24 h after the challenge the swelling was nearly abolished by AWD 12-281 (FIG. 4).

Histological Examination

The histological examination of the mouse ear skin 24 h after the TDI challenge shows a distinct edema and an influx of inflammatory cells (mainly granulocytes). AWD 12-281, cilomilast and diflorasone inhibited these inflammatory processes markedly (Table 1).

Inflammatory Mediators in the Mouse Skin

The concentration of the cytokines are generally higher in ears of mice rechallenged five weeks after the first treatment. This is obviously due to residues of inflammatory cells in ear skin. So these results (TDI compared to AWD 12-281 3%) are presented seperately in Table 2).

There was a significant increase of IL-4 in TDI treated mouse skin 24 h after the challenge (FIG. 5a). 1% AWD 12-281 showed a slight inhibitory effect, whereas 3% AWD 12-281 (Table 2), cilomilast (3%) and diflorasone (0.05%) inhibited the increase significantly.

The concentration of IL-6 was also significantly increased by TDI 24 h after challenge. AWD 12-281 (1%, 3%) cilomilast (3%) and diflorasone (0.05%) inhibited this response significantly. The inhibitory effect of AWD 12-281 (3%), cilomilast and diflorasone was comparable, while AWD 12-281 (1%) showed only a slight effect (FIG. 5b and Table 2).

MIP-2, a functional homologue of human IL-8, was also increased after TDI challenge. Whereas 1% AWD 12-281 reduced this increase slightly, 3% AWD 12-281, cilomilast (3%) and diflorasone (0.05%) diminished the increase of the MIP-2 concentration significantly (FIG. 5c and Table 2).

3. Discussion

The study was performed to investigate the effect of PDE4 inhibitors on the TDI induced mouse ear swelling. TDI, administered to skin induces a predominantly Th2-type cytokine mediated reaction, demonstrated by high amounts of interleukin 4 and 10 in activated lymph node cells from TDI exposed animals but only low levels of interferon α (Dearman et al., 1996, Hayashi et al., 2001). Our own results indicate that additional cytokines like IL-1β, IL-6 and MIP-2 are also involved in the allergic/inflammatory skin reaction of TDI. The increase of proinflammatory cytokines is accompanied by an influx of inflammatory cells like neutrophils and eosinophils (Table 1) and a distinct edema. Therefore, the ear swelling is useful as a functional parameter.

A skin permeability test of AWD 12-281 was performed. In our experiment, AWD 12-281 was very well absorbed and could penetrate into the buffer. The observed cumulative absorption of 0.08 and 0.22% respectively after 6 h amounts to a tenfold higher absorption compared to hydrocortisone, measured for human skin in Franz diffusion cells (Hueber et al., 1994).

The results concerning topical treatment of the PDE4 inhibitors before TDI challenge confirm former findings (Ehinger et al., 2000), In this study AWD 12-281 was administered in a lower dose to take the different IC₅₀s for PDE4 into account. The IC₅₀ of AWD 12-281 is about ten times lower than that of cilomilast (Griswold et al., 1998, Kuss et al., 2002).

In contrast to former results, it was possible to obtain a more distinct positive control by modifying the sensitization protocol. By repeated use of the sensitization protocol described in this study, it was possible to reproduce, and standardize the high response to TDI 24 h after challenge (data not shown). Due to the high response at that time point, we were able to detect high amounts of cytokines. To get closer to clinical circumstances, it was decided to examine effects of the PDE4 inhibitors in treating a allergic/inflammatory reaction which set already in. Therefore the PDE4 inhibitors were given 1 h after TDI challenge where a beginning of inflammatory processes is reflected by a mean ear swelling of 30%. Although administered at a lower dose (1%) AWD 12-281 reduced the inflammatory response to TDI significantly 24 h after the challenge. This was confirmed by the study, where the effects of a long term exposure to TDI was examined. One effect of this long term exposure was an elevated ear swelling 1 h after challenge (FIG. 4) which might be due to a more IgE mediated response (Scheerens et al., 1999). Administered at a higher concentration (3%) AWD 12-281 nearly abolished the TDI induced swelling in this long term exposure study (FIG. 4). The histological examination of the AWD 12-281 treated mouse ears showed—in vast contrast to the positive control—a nearly total absense of inflammatory cells and vascular leakage (not shown).

To examine inflammatory mediators responsible for the TDI induced ear swelling, the cytokines interleukin (IL) 4, IL-6 and MIP-2 were measured in treated mouse ears.

An IL-4 overproduction is apparent in acutely affected skin lesions of patients suffering from atopic dermatitis (Hanifin et al., 1996, Spergel et al., 1999). So we were interested in the influence of TDI on IL-4 production in mouse skin. The source for IL-4 in the skin is limited, as keratinocytes and Langerhans cells do not produce this cytokine (Shreedhar et al., 1998, Morita et al., 2001). Therefore it is of interest that such an immense effect is registered in the positive control. Mast cells (Harvima et al., 1994; Dastych et al., 1999) and the influx of Th₂ cells (Shreedhar et al., 1998) are obviously the source of IL-4 in the skin. The ability of cilomilast to inhibit IL-4 in vivo has also been demonstrated in a model of chronic oxazolone-induced contact sensitivity (Griswold et al., 1998). The insufficient modulatory effect by AWD 12-281 (1%) is due to the lower dose, as 3% AWD 12-281 results in 80% inhibition of IL-4 concentration (Table 2B).

IL-6 is described as being elevated through TDI in vitro (Mattoli et al., 1991). IL-6 is secreted by keratinocytes after an inflammatory stimulus (McKenzie et al., 1990). An inhibition of IL-6 release by PDE4 inhibitors is also described for LPS stimulated macrophages (Kambayashi et al., 1995).

MIP-2 is a crucial cytokine for the chemotaxis of neutrophils. Demonstrated here TDI provoked ear swelling goes along with a vast influx of neutro-phils. The inhibitory effect of cilomilast, diflorasone and AWD 12-281 (3%) may explain the reduced influx of neutrophils after treatment with a PDE4 inhibitor or a glucocorticoid.

Taken together, these results suggest that AWD 12-281, as well as cilomilast can inhibit inflammatory reactions in a model of allergic dermatitis. The antiinflammatory response of AWD 12-281 is reliable given via the topical route and there is obviously an improved inhibition by administration of 3% compared to 1% (FIGS. 3 and 4). Although cilomilast (3%) has also inhibitory effects via the oral and intraperitoneal route it lacks significant inhibitory effects when administered after the TDI challenge (FIG. 3). Taking into account that the treatment of allergic reactions is clinically more important than a preventive administration, these data indicate an advantage of AWD 12-281 and related hydroxy indole compounds in the treatment of skin diseases, particularly allergic/inflammatory reactions in the skin.

TABLE 1
Histological examination of cell influx in mice ears 24 h
after TDI challenge. Tissue samples were taken from the
experiment with topical treatment (see FIG. 2).

| | | | toluene-2,4-diisocyanate | | |
|---|---|---|---|---|---|
| | untreated | | AWD 12- | cilomilast | diflorasone |
| | control | vehicle | 281 (1%) | (3%) | (0.05%) |
| Granulocyte | · | +++ | + | −/+ | −/+ |
| score in the | | | | | |
| dermis | | | | | |

TABLE 2
A)
Challenge with TDI

| | vehicle | AWD 12-281 |
|---|---|---|
| | (control) | (3%) |
| Ear swelling (%) | 51 ± 7 | 1 ± 2** |
| IL-4 | 67 ± 2 | 12 ± 3** |
| IL-6 | 89 ± 5 | 42 ± 6** |
| MIP-2 | 286 ± 39 | 72 ± 8** |

B)

| Cytokine | AWD 12-281 (3%) | cilomilast (3%) | diflorasone (0.05%) |
|---|---|---|---|
| IL-4 | 81 | 74 | 50 |
| IL-6 | 52 | 49 | 52 |
| MIP-2 | 75 | 66 | 76 |

A) Ear swelling 24 h after TDI challenge and mean values (±s.e. mean) of IL-4, IL-6 and MIP-2 (μg/400 pg protein) in homogenized mouse ears 24 h after TDI challenge after treatment with AWD 12-281 (3%),
**P < 0.01.
B) Comparison of the inhibitory effect (mean inhibition (%) of the TDI induced synthesis) of AWD 12-281 (3%), cilomilast (3%) and diflorasone (0.05%).

References

BUTLER, J. M., CHAN, S. C., STEVENS, S. R. & HANIFIN, J. M. (1983). Increased leukocyte histamine release with elevated cyclic AMP-phosphodiesterase activity in atopic dermatitis. J. Allergy Clin. Immunol. 71, 490-497.

COOPER, K. D., KANK, K., CHAN, S. C. & HANIFIN, J. M. (1985). Phosphodiesterase inhibition by Ro 20-1724 reduces hyper-IgE synthesis by atopic dermatitis cells in vitro. J. Invest. Dermatol. 84, 477-482.

DASTYCH, J., WALCZAK-DRZEWIECKA, A., WYCZOLKOWSKA, J. & METCALFE, D. D. (1999). Murine mast cells exposed to mercuric chloride release granule-associated N-acetyl-beta-D-hexosaminidase and secrete IL-4 and TNF-alpha. J. Allergy Clin. Immunol. 103, 1108-1114.

DEARMAN, R. J., BASKETTER, D. A. & KIMBER, I (1996). Characterisation of chemical allergens as a function of divergent cytokine secreteion profiles induced in mice. Toxicol. Appl. Pharmacol. 138, 308-316

DENT, G., GIEMBYCZ, M. A., EVANS, P. M., RABE, K. F. & BARNES, P. J. (1994). Suppression of human eosinophil respiratory burst and cyclic AMP hydrolysis by inhibitors of type IV phosphodiesterase: interaction with the beta adrenoreceptor agonist albuterol. J. Pharmacol. Exp. Ther. 271, 1167-1174.

DYKE, H. J. & MONTANA, J. G. (2002): Update on the therapeutic potential of PDE4 inhibitors. Expert. Opin. Investig. Drugs 11, 1-13

HINGER, A. M., GORR, G., HOPPMANN, J., TELSER, E., EHINGER, B. & KIETZMANN, M. (2000). Effects of the phosphodiesterase 4 inhibitor RPR 73401 in a model of immunological inflammation. Eur. J. Pharmacol, 392, 93-99.

ENK, A. H. & KATZ, S. I. (1992). Early molecular events in the induction phase of contact sensitivity, Proc. Natl. Acad. Sci. USA 89, 1398-1402.

EZEAMUZIE, C. I. (2001). Requirement of additional adenylate cyclase activation for the inhibition of human eosinophil degranulation by phosphodiesterase IV inhibitors. Eur. J. Pharmacol. 417, 11-18.

GAD, S. C., DUNN, B. J., DOBBS, D. W., REILLY, C. &WALSH, R. D. (1986). Development and validation of an alternative dermal sensitization test: the mouse ear swelling test (MEST). Toxicol. Appl. Pharmacol. 84, 93-114.

GANTNER, F., KUPFERSCHMIDT, R., SCHUDT, C., WENDEL, A. & HATZELMANN, A. (1997). In vitro differentiation of human monocytes to macrophages: change of PDE profile and its relationship to suppression of tumor necrosis factor (release by PDE inhibitors. Br. J. Pharmacol. 121, 221-231.

GIEMBYCZ, M. A. (2000). Phosphodiesterase 4 inhibitors and the treatment of asthma: where are we now and where do we go from here? Drugs 59, 193-212.

GIEMBICZ, M. A. (2001). Cilomilast: a second generation phosphodiestersa 4 inhibitor for asthma and chronic obstructive pulmonary disease. Expert Opin. Investig. Drugs 10, 1361-1379

GRISWOLD, D. E., WEBB, E. F., BADGER, A. M., GORYCKI, P. D., LEVANDOSKI, P. A., BARNETTE, M. A., GROUS, M., CHRISTENSEN, S. & TORPHY, T. J. (1998). SB 207499 (Ariflo), a second generation phosphodiesterase 4 inhibitor, reduces tumor necrosis factor (and interleukin-4 production in vivo J. Pharmacol, Exp. Therap. 287, 705-711.

HANIFIN, J. M., CHAN, S. C., CHENG, S. B., TOFTE, S. J., HENDERSON, W. R., KIRBY, D. S. & WEINER, E. S. (1996). Type 4 phosphodiesterase inhibitors have clinical and in vitro anti-inflammatory effects in atopic dermatitis. J. Invest. Dermatol. 107, 51-56.

HARVIMA, I. T., HORSMANHFIMO, L., NAUKKARINEN, A. & HORSMANHEIMO, M. (1994). Mast cell proteinase and cytokines in skin inflammation. Arch. Dermatol, Res. 287, 61-67.

HATZELMANN, A. & SCHUDT, C. (2001). Anti-inflammatory and immunomodulatory potential of the novel PDE4 inhibitor roflumilast in vitro. J. Pharmacol. Exp. Therap. 297, 267-279

HAYASHI, M., HIGASHI, K., KATO, H. & KANEKO, H. (2001). Assessment of prefential Th1 or Th2 induction by low-molecular-weight compounds unsing a reverse transkription-polymerase chain reaction method: Comparison of two mouse strains, C57BL/6 and Balb/c. Toxicol. Appl. Pharmacol. 177, 38-45

HEER, S., KUESTERS, S. & SZELENYI, I. (1999). In-vitro effect of the selective phosphodiesterase 4 inhibitor AWD 12-281 and of glucocorticoids on lipopolysaccharide (LPS)-induced TNF(-release in diluted and undiluted human blood of healthy volunteers. N.-S. Archiv Pharmacal. 359 (Suppl. 3), 327.

HUEBER, F., SCHAEFER, H. & WEPIERRE, J. (1994). Role of trans-epidermal and transfollicular routs in percoutaneous absorption of steroids: In vitro studies an human skin. Skin Pharmacol. 7, 237-244

KAMBAYASHI, T., JAKOB, C. O., ZHOU, D., MAZUREK, N., FONG, M. & STRASSMANN, G. (1995). Cyclic nucleotide phosphodiesterese type IV participates in the regulation of IL-10 and in the subsequent inhibition of TNF-(and IL-6 release by endotoxin-stimulated macrophages. J. Immunol. 155, 4909-4916.

KAROL, M. H., TOLLERUD, D. J., CAMPBELL, T. P., FABBRI, L., MAESTRELLI, P., SAETTA, M. & MAPP, C. E. (1994). Predictive value of airways hyperresponsiveness and circulating IgE for identifying types of responses to toluene diisocyanate inhalation challenge. Am. J. Respir. Crit. Care Med. 149, 611-615.

KONDO, S., PASTORE, S., SHIVJI, G. M., MCKENZIE, R. C. & SAUDER, D. N. (1994). Characterisation of epidermal cytokine profiles in sensitization and elicitation phases of allergic contact dermatitis as well as irritant contact dermatitis. Lymphok. Cytok. Res. 13, 367-375

KUSS, H., HÖFGEN, N., EGERLAND, U., HEER, S., MARX, D., SZELENYI, I., SCHUPKE, A., GASPARIC, M., OLBRICH, M., HEMPEL, R., HARTENHAUER, H., KRONE, D., BERTHOLD, K., KRONBACH, T. & RUNDFELDT, C. (2002). AWD 12-281. Drug Future 27, 111-116

MAESTRELLI, P., DI STEFANO, A., OCCARI, P., TURATO, G., MILANI, G., PIVIROTTO, F., MAPP, C. E., FABBRI, L. M. & SAETTA, M. (1995). Cytokines in the airway mucosa of subjects with asthma induced by toluene diisocyanate. Am. J. Respir. Crit. Care Med. 151, 607-612.

MCKENZIE, R. C. & SAUDER, D. N. (1990). The role of keratinocyte cytokines in inflammation and immunity. J. Invest. Dermatol. 95 (Suppl.) 105S-107S.

SCHEERENS, H., BUCKLEY, T. L., MUIS, T. L., GARSSEN, J., DORMANS, J., NIJKAMP, F. P. & VAN LOWEREN, H. (1999). Long-term topical exposure to toluene diisocyanate in mice leads to antibody production and in vivo airway hyperresponsiveness three hours after intranasal challenge. Am. J. Respir. Crit. Care Med. 159, 1074-1080.

SCHMIDT, D. T., WATSON, N., DENT, G., RÜHLMANN, E., BRANSCHEID, D., MAGNUSSEN, H. & RABE, K. F, (2000). The effect of selective and non-selective phosphodiesterase inhibitors on allergen- and leukotriene C4-induced contractions in passively sensitized human airways. Br. J. Pharmacol. 131, 1607-1618.

SIKORSKI, E. E., GERBERICK, G. F., LIMARDI, L. C. (1997). Evaluation of cytokine message in the EpiDerm in vitro skin model following application of contact allergens and skin irritants, J. Invest. Dermatol. 108, 662

SPERGEL, J. M., MIZOGUSCHI, E., OETTGEN, H., BHAN, A. K. & GEHA, R. S. (1999). Roles of TH1 and TH2 cytokines in a murine model of dermatitis. J. Clin. Invest. 103, 1103-1111.

TOFOVIC, S. P., ZACCHARIA, L. C., CARCILLO, J. A. & JACKSON, E. K. (2000). Inhibition of cytokine release by and cardiac effects of type IV phosphodiesterase inhibition in early, profound endotoxaemia in vivo. Clin. Exp. Pharmacol. Physiol. 27, 787-792.

TOMINAGA, M., KOHNO, S., TANAKA, K. & OHATA, K. (1985). Studies on toluene diisocyanate (TDI)-induced delayed type hypersensitivity. Jpn. J. Pharmacol. 39, 163-171.

VERGHESE, M. W., MCCONNELL, R. T, STRICKLAND, A. B., GOODING, R. C., STIMPSON, S. A., YARNALL, D. P., TAYLOR, J. D. & FURDON, P. J. (1995). Differential regulation of human monocyte-derived TNF(and IL-1(by type IV cAMP-phosphodiesterase (cAMP-PDE) inhibitors. J. Pharmacol. Exp. Ther. 272, 1313-1320.

1. A method for the treatment of a skin disease comprising topically administering a subject in need thereof a therapeutically effective amount of a compound of formula (I) or a pharmaceutically acceptable salt thereof: 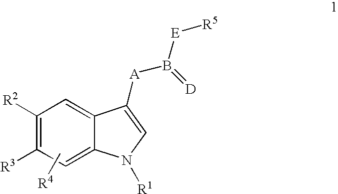
in which
R¹ is
(i) —C₁₋₁₂-alkyl, straight-chain or branched-chain or —C₂-C₁₂ alkenyl, mono- or polyunsaturated,
optionally mono- or polysubstituted by —OH, —SH, —NH₂, —NHC₁₋₆-alkyl, —N(C₁₋₆-alkyl)₂, —NHC₆₋₁₄aryl, —N(C₆₋₁₄aryl)₂, —N(C₁₋₆alkyl)(C₆₋₁₄aryl), —NHCOR⁶, —NO₂, —CN, —F, —Cl, —Br, —I, —O—C₁₋₆-alkyl, —O—C₆₋₁₄-aryl, —O(CO)R⁶, —S—C₁₋₆-alkyl, —S—C₆₋₁₄aryl, —SOR⁶, —SO₃H, —SO₂R⁶, —OSO₂C₁₋₆alkyl, —OSO₂C₆₋₁₄aryl, —(CS)R⁶, —COOH, —(CO)R⁶, mono-, bi- or tricyclic saturated or mono- or polyunsaturated carbocycles having 3-14 ring members, mono-, bi- or tricyclic saturated or mono- or polyunsaturated heterocycles having 5-15 ring members and 1-6 heteroatoms, which are preferably N, O and S, wherein the C₆₋₁₄aryl groups and the carbocyclic and heterocyclic substituents for their part can optionally be mono- or polysubstituted by R⁴,
(ii) a mono-, bi- or tricyclic saturated or mono- or polyunsaturated carbocycle having 3-14 ring members or a mono-, bi- or tricyclic saturated or mono- or polyunsaturated heterocycle having 5-15 ring members and 1-6 heteroatoms, which are preferably N, O and S, or a carbo- or heterocyclic saturated or mono- or polyunsaturated spirocycle having 3-10 ring members, where heterocyclic systems contain 1-6 heteroatoms, which are preferably N, O and S, optionally mono- or polysubstituted by —OH, —SH, —NH₂, —NHC₁₋₆-alkyl, —N(C₁₋₆-alkyl)₂, —NHC₆₋₁₄aryl, —N(C₆₋₁₄aryl)₂, —N(C₁₋₆alkyl)(C₆₋₁₄aryl), —NHCOR⁶, —NO₂, —CN, —F, —Cl, —Br, —I, —O—C—₁₋₆-alkyl, —O—C₆₋₁₄-aryl, —O(CO)R⁶, —S—C₁₋₆-alkyl, —S—C₆₋₁₄aryl, —SOR⁶, —SO₃H, —SO₂R⁶, —OSO₂C₁₋₆alkyl, —OSO₂C₆₋₁₄aryl, —(CS)R⁶, —COOH, —(CO)R⁶, mono-, bi- or tricyclic saturated or mono- or polyunsaturated carbocycles having 3-14 ring members, mono-, bi- or tricyclic saturated or mono- or polyunsaturated heterocycles having 5-15 ring members and 1-6 heteroatoms, which are preferably N, O and S, wherein the C₆₋₁₄aryl groups and the carbocyclic and heterocyclic substituents can optionally be mono- or polysubstituted by R⁴,
R⁵ is
a mono-, bi- or tricyclic saturated or mono- or polyunsaturated carbocycle having 3-14 ring members or a mono-, bi- or tricyclic saturated or mono- or polyunsaturated heterocycle having 5-15 ring members and 1-6 heteroatoms, which are preferably N, O and S, or a carbo- or heterocyclic saturated or mono- or polyunsaturated spirocycle having 3-10 ring members, where heterocyclic systems contain 1-6 heteroatoms, which preferably N, O and S, optionally mono- or polysubstituted by —OH, —SH, —NH₂, —NHC₁₋₆-alkyl, —N(C₁₋₆-alkyl)₂, —NHC₆₋₁₄aryl, —N(C₆₋₁₄aryl)₂, —N(C₁₋₆alkyl)(C₆₋₁₄aryl), —NHCOR⁶, —NO₂, —CN, —F, —Cl, —Br, —I, —O—C—₁₋₆-alkyl, —O—C₆₋₁₄-aryl, —O(CO)R⁶, —S—C₁₋₆-alkyl, —S—C₆₋₁₄aryl, —SOR⁶, —SO₃H, —SO₂R⁶, —OSO₂C₁₋₆alkyl, —OSO₂C₆₋₁₄aryl, —(CS)R⁶, —COOH, —(CO)R⁶, mono-, bi- or tricyclic saturated or. mono- or polyunsaturated carbocycles having 3-14 ring members, mono-, bi- or tricyclic saturated or mono- or polyunsaturated heterocycles having 5-15 ring members and 1-6 heteroatoms, which are preferably N, O and S, wherein the C₆₋₁₄aryl groups and the carbocyclic and heterocyclic substituents can optionally be mono- or polysubstituted by R⁴, with the proviso that R⁵ contains at least one substituent selected from —F, —Cl, —Br, —I;
R², R³ are hydrogen or —OH, where at least one of the two substituents must be —OH;
R⁴ is
—H, —OH, —SH, —NH₂, —NHC₁₋₆-alkyl, —N(C₁₋₆-alkyl)₂, —NHC₆₋₁₄aryl, —N(C₆₋₁₄aryl)₂, —N(C₁₋₆alkyl) (C₆₋₁₄aryl), —NHCOR⁶, —NO₂, —CN, —COOH, —(CO)R⁶, —(CS)R⁶, —F, —Cl, —Br, —I, —O—C₁₋₆-alkyl, —O—C₆₋₁₄-aryl, —O(CO)R⁶, —S—C₁₋₆-alkyl, —S—C₆₋₁₄aryl, —SOR⁶, —SO₂R⁶, —C₁-C₆-alkyl,
wherein each aryl or alkyl may be mono- or polysubstituted by —OH, —F, —Cl, —Br, —I;
R⁶ is
—H, —NH₂—NHC₁₋₆-alkyl, —N(C₁₋₆-alkyl)₂, —NHC₆₋₁₄aryl, —N(C₆₋₁₄aryl)₂—N(C₁₋₆alkyl) (C₆₋₁₄aryl), —O—C₁₋₆-alkyl, —O—C₆₋₁₄-aryl, —S—C₁₋₆-alkyl —S—C₆₋₁₄aryl, —C₁₋₁₂-alkyl, straight-chain or branched-chain,
—C₂₋₁₂-alkenyl, mono- or polyunsaturated, straight-chain or branched-chain,
mono-, bi- or tricyclic saturated or mono- or polyunsaturated carbocycles having 3-14 ring members,
mono-, bi- or tricyclic saturated or mono- or polyunsaturated heterocycles having 5-15 ring members and 1-6 heteroatoms, which are preferably N, O and S;
A is either a bond, or
—(CH₂)ₘ—, —(CH₂)ₘ—(CH═CH)ₙ(—CH₂)ₚ—, —(CHOZ)ₘ—, —(C═O)—, —(C═S)—, —(C═N—Z)—, —O—, —S—, —NZ—,
wherein m, p=0-3 and n=0-2 and
Z is
—H, or
—C₁₋₁₂-alkyl, straight-chain or branched-chain,
—C₂₋₁₂-alkenyl, mono- or polyunsaturated, straight-chain or branched-chain,
mono-, bi- or tricyclic saturated or mono- or polyunsaturated carbocycles having 3-14 ring members,
mono-, bi- or tricyclic saturated or mono- or polyunsaturated heterocycles having 5-15 ring members and 1-6 heteroatoms, which are preferably N, O and S;
B is either carbon or sulfur, or —(S═O)—;
D is oxygen sulfur, CH₂ or N—Z,
where, if B is carbon, D is S or CH₂;
E is a bond, or
—(CH₂)ₘ—, —O—, —S—, —(N═Z)—, wherein m and Z have the meaning already described above,

2. The method of claim 1 wherein R⁵ is selected from monocyclic saturated or mono- or polyunsaturated carbocycles and heterocycles having at least one halogen substituent.

3. The method of claim 2 wherein R⁵ is is selected from monocyclic aromatic carbocycles and heterocycles having at least one halogen substituent.

4. The method of claim 3 wherein R⁵ is a pyridine ring having at least one halogen substituent.

5. The method of claim 3 wherein R⁵ is a phenyl ring having at least one halogen substituent.

6. The method of claim 1 wherein R¹ is selected from C₁-C₁₂ alkyl, which is optionally substituted.

7. The method of claim 1 wherein R¹ is selected from monocyclic saturated or mono- or polyunsaturated carbocycles or heterocycles, which are optionally substituted.

8. The method of claim 1 wherein R² is OH and R³ is H.

9. The method of claim 1 wherein A is selected from —(C═O)— and —(CHOH)—.

10. The method of claim 1 wherein B is C.

11. The method of claim 1 wherein D is O.

12. The method of claim 1 wherein E is —(N—H)—.

13. The method of claim 1 wherein compound (I) is (N-3,5-dichloro-4-pyridinyl)-2-[1-(4-fluorobenzyl)-5-hydroxy-1 H-indol-3-yl]-2-oxoacetamide).

14. The method of any one of claims 1-13 wherein the skin disease is an allergic and/or inflammatory disease.

15. The method of claim 14 wherein the allergic disease is allergic dermatitis.

16. The method of any one of claims 1-15 wherein the compound is administered to a skin area which is afflicted by disease.

17. The method of claim 16 wherein the compound is administered after an allergic challenge.

18. The method of claim 17 wherein the compound is administered up to 48 h after the allergic challenge.

19. The method of any one of claims 1-18 wherein the compound (I) is co-administered with at least one further pharmaceutical agent.

20. The method of claim 19 wherein the further pharmaceutical agent is a drug stimulating cAMP production.

21. The method of claim 20 wherein the further pharmaceutical agent is a corticosteroid.
